SEQUENCE LISTING (19 sequences)

SEQ ID NO: 1 - protein, 448 aa, Streptococcus sp.
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGIKDLQAQVVESAKKARISEATDGLSDFLKSQTPAEDTVKSIELAEAKVLANRELDKYGVSDYHKNLINNAKTVEGVKELIDEILAALPKTDTYKLILNGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKDDAKKAETLPTTGEGSNPFFTAAALAVMAGAGALAVASKRKED

SEQ ID NO: 2 - protein, 41 aa, Streptococcus sp.
TYKLILNGKTLKGETTTEAVDAATAEKVFKQYANDNGVDGE

SEQ ID NO: 3 - DNA, 1950 bp, Streptococcus sp.
aagctttggtggagaaattggctggcgaatccagcttcaccggtgtttcaccagtagatgctttctgtggtcttattgacacgcacttgtggcgagagtactaacagtcacagcgacgttaactttattttccttatgagaggttaagaaaaaacgttattaaatagcagaaaagaatattatgactgacgttaggagttttctcctaacgttttttttagtacaaaaagagaattctctattataaataaaataaatagtactatagatagaaaatctcatttttaaaaagtcttgttttcttaaagaagaaaataattgttgaaaaattatagaaaatcatttttatactaatgaaatagacataaggctaaattggtgaggtgatgataggagatttatttgtaaggattccttaattttattaattcaacaaaaattgatagaaaaattaaatggaatccttgatttaattttattaagttgtataataaaaagtgaaattattaaatcgtagtttcaaatttgtcggctttttaatatgtgctggcatattaaaattaaaaaaggagaaaaaatggaaaaagaaaaaaaggtaaaatactttttacgtaaatcagcttttgggttagcatccgtatcagctgcatttttagtgggatcaacggtattcgctgttgattcaccaatcgaagataccccaattattcgtaatggtggtgaattaactaatcttctggggaattcagagacaacactggctttgcgtaatgaagagagtgctacagctgatttgacagcagcagcggtagccgatactgtggcagcagcggcagctgaaaatgctggggcagcagcttgggaagcagcggcagcagcagatgctctagcaaaagccaaagcagatgcccttaaagaattcaacaaatatggagtaagtgactattacaagaatctaatcaacaatgccaaaactgttgaaggcataaaagaccttcaagcacaagttgttgaatcagcgaagaaagcgcgtatttcagaagcaacagatggcttatctgatttcttgaaatcgcaaacacctgctgaagatactgttaaatcaattgaattagctgaagctaaagtcttagctaacagagaacttgacaaatatggagtaagtgactatcacaagaacctaatcaacaatgccaaaactgttgaaggtgtaaaagaactgatagatgaaattttagctgcattacctaagactgacacttacaaattaatccttaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaaccagaagtgatcgatgcgtctgaattaacaccagccgtgacaacttacaaacttgttattaatggtaaaacattgaaaggcgaaacaactactaaagcagtagacgcagaaactgcagaaaaagccttcaaacaatacgctaacgacaacggtgttgatggtgtttggacttatgatgatgcgactaagacctttacggtaactgaaatggttacagaggttcctggtgatgcaccaactgaaccagaaaaaccagaagcaagtatccctcttgttccgttaactcctgcaactccaattgctaaagatgacgctaagaaagacgatactaagaaagaagatgctaaaaaaccagaagctaagaaagatgacgctaagaaagctgaaactcttcctacaactggtgaaggaagcaacccattcttcacagcagctgcgcttgcagtaatggctggtgcgggtgctttggcggtcgcttcaaaacgtaaagaagactaattgtcattatttttgacaaaaagctt

SEQ ID NO: 4 - DNA, 123 bp, Streptococcus sp.
acttacaaattaatccttaatggtaaaacattgaaaggcgaaacaactactgaagctgttgatgctgctactgcagaaaaagtcttcaaacaatacgctaacgacaacggtgttgacggtgaa

SEQ ID NO: 5 - protein, 183 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 6 - protein, 145 aa, Streptomyces avidinii
AGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 7 - DNA, 638 bp, Streptomyces avidinii
ccctccgtccccgccgggcaacaactagggagtatttttcgtgtctcacatgcgcaagatcgtcgttgcagccatcgccgtttccctgaccacggtctcgattacggccagcgcttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtagtcgcgtcccggcaccggcgggtgccgggacctcggcc

SEQ ID NO: 8 - DNA, 435 bp, Streptomyces avidinii
gccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcag

SEQ ID NO: 9 - DNA, 27 bp, Artificial Sequence
  PCR primer
catatgcacttacaaattaatccttaa

SEQ ID NO: 10 - DNA, 31 bp, Artificial Sequence
  PCR primer
gaattcggatccttcaccgtcaacaccgttg

SEQ ID NO: 11 - DNA, 32 bp, Artificial Sequence
  PCR primer
gaattcaagcttgccggcatcaccggcacctg

SEQ ID NO: 12 - DNA, 26 bp, Artificial Sequence
  PCR primer
ctgcagctgctgaacggcgtcgagcg

SEQ ID NO: 13 - protein, 301 aa, Artificial Sequence
  fusion protein
MTMITPSLVPSSDPLVTAASVLEFGFICTYKLILNGKTLKGETTTEAVDAATAEKVFKQYANDNGVDGEGSEFKLAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQLQKPNSADIHHTGGRSSMHLEGPIRPIVSRITIHWPSFYNVVTGKTLALPNLIALQHIPLSPAGVIAKRPAPIALPNSCAA

SEQ ID NO: 14 - DNA, 906 bp, Artificial Sequence
  DNA encoding the fusin protein
atgaccatgattacgccaagcttggtaccgagctcggatccactagtaacggccgccagtgtgctggaattcggcttcatatgcacttacaaattaatccttaatggtaaaacattgaaaggcgaaacaactactgaagctgttgatgctgctactgcagaaaaagtcttcaaacaatacgctaacgacaacggtgttgacggtgaaggatccgaattcaagcttgccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagctgcagaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctga

SEQ ID NO: 15 - DNA, 131 bp, Artificial Sequence
  Oligo #1
cactgcttactggcttatcgaaatggaattctgcatgcatctagagggccctattctatagcatagtgtcacctaaatgctaggcaccttctagttgccagccatctgttgcacaccaaacgtggcttgcc

SEQ ID NO: 16 - DNA, 23 bp, Artificial Sequence
  PCR primer
cactgcttactggcttatcgaaa

SEQ ID NO: 17 - DNA, 18 bp, Artificial Sequence
  PCR primer
ggcaagccacgtttggtg

SEQ ID NO: 18 - DNA, 161 bp, Artificial Sequence
  Oligo #7
cactgcttactggcttatcgaaatggaattctgcatgcatctagagggccctattctatagcatagtgtcacctaaatgctaggcaaccgacaattgcatgaagaactcgcacattgacgtcaataatgacgtatgttcccaccaccaaacgtggcttgcc

SEQ ID NO: 19 - DNA, 24 bp, Artificial Sequence
  PCR probe
ccttctagttgccagccatctgtt

(19) United States
(12) Patent Application Publication
(10) Pub. No.: US 2016/0069879 A1
(43) Pub. Date: Mar. 10, 2016

(54) METHOD FOR MEASURING ALPHA-GALACTOSIDASE CONCENTRATION

(71) Applicants: TOKYO METROPOLITAN ORGANIZATION FOR MEDICAL RESEARCH, Tokyo (JP); MEIJI PHAMACEUTICAL UNIVERSITY, Tokyo (JP)

(72) Inventors: Hitoshi SAKURABA, Chiba (JP); Futoshi SHIBASAKI, Tokyo (JP); Sachie NAKANO, Tokyo (JP)

(21) Appl. No.: 14/433,418

(22) PCT Filed: Nov. 27, 2013
(86) PCT No.: PCT/IB2013/002652

§ 371 Date: Oct. 28, 2015

(30) Foreign Application Priority Data
Oct. 4, 2012 (JP) JP2012-222578

(51) Int. Cl. G01N 33/573

(52) U.S. Cl. CPC G01N 33/573; G01N 2333/94; G01N 2800/7085

(57) ABSTRACT

The purpose of this invention is to provide novel methods of measuring concentrations of α-galactosidase in blood, serum, plasma, cells, or tissues. For solution of this purpose, concentrations of α-galactosidase are measured using a MUSTag method in the blood, serum, plasma, cells, or tissues.

### TECHNICAL FIELD

The present invention relates to methods of measuring a concentration of α-galactosidase.

### BACKGROUND ART

Fabry disease is a genetic disease that allows glycolipids that would be normally degraded by a lysosomal enzyme, α-galactosidase (GLA), to be systemically accumulated and causes pain, kidney failure, heart failure, and cerebrovascular disorders due to significant reduction of the activity of the enzyme.

This disease has an X-linked pattern of inheritance. In male Fabry hemizygotes, α-galactosidase activity decreases in all body cells, which directly reflects in the clinical picture of the disease, usually resulting in severe symptoms. However, detail analyses of a group of male patients with Fabry disease have shown that in addition to patients with classic Fabry disease who have severe manifestations of the symptoms resulting from an almost complete absence of intracellular α-galactosidase activity due to deletions, insertions, nonsense mutations, or even some missense or splicing mutations of the α-galactosidase gene, there are patients with atypical Fabry disease who have residual α-galactosidase activity and mild manifestations of the symptoms due to other missense or splicing mutations. The patients with classic Fabry disease have disorders of various organs in the body, while those with atypical Fabry disease have disorders of a specific organ mainly in the heart or kidney. Atypical Fabry disease is thus referred to as cardiac variant of Fabry disease or renal variant of Fabry disease according to the affected organ.

On the other hand, in female Fabry heterozygotes, groups of cells with decreased α-galactosidase activity and those with normal α-galactosidase activity are present in a mosaic fashion throughout their body due to random X-inactivation characteristic of X-linked inheritance. Accordingly, clinical pictures of the disease vary significantly depending on the percentage of affected and unaffected groups of cells, ranging from patients having a severity comparable to those with the classic Fabry disease to patients who are almost asymptomatic. Although the number of female Fabry homozygotes is small, they have symptoms similar to those observed in male Fabry hemizygotes.

Conventionally, the quantity of α-galactosidase protein in clinical samples such as serum or plasma has been determined using methods such as ELISA. Fuller et al. reported that the concentrations of α-galactosidase in blood spotted on a filter paper could be used for distinguishing Fabry hemizygotes from wild-type controls (Fuller M, Lovejoy M, Brooks D A, Harkin M L, Hopwood J J, Meikle P: Immunoquantification of α-galactosidase: Evaluation for the diagnosis of Fabry disease. Clin Chem, 50:1979-1985, 2004). Fabry heterozygotes, however, could be distinguished from neither the wild-type controls nor the Fabry hemizygotes by using this method. Only after evaluating the ratio of saposin C and α-galactosidase concentrations, the Fabry heterozygotes could be distinguished from wild-type controls. By using plasma samples, it was possible to distinguish Fabry hemizygotes and hetero zygotes from wild-type controls, but Fabry hemizygotes and heterozygotes could not be distinguished from one another.

An object of the present invention is to provide novel methods of measuring concentrations of α-galactosidase in blood, serum, plasma, cells, or a tissue.

### SUMMARY OF THE INVENTION

An aspect of the present invention is a method of measuring a concentration of α-galactosidase in blood, serum, or plasma, comprising the step of measuring the concentration of α-galactosidase using a MUSTag method. The method of the invention may further comprise the step of determining, using the measured concentration of α-galactosidase, whether the blood, the serum, the plasma, the cell, or the tissue is originated from a patient with classic Fabry disease, a patient with atypical Fabry disease or a healthy individual. In addition, a concentration of saposin C may not be measured in the blood, the serum, the plasma, the cell, or the tissue. Furthermore, α-galactosidase activity may not be measured in the blood, the serum, the plasma, the cell, or the tissue.

The present invention can provide novel methods of measuring concentrations of α-galactosidase in blood, serum, plasma, cells, or a tissue.

### BRIEF DESCRIPTION OF THE DRAWINGS

FIG. 1 is a graphical representation of a calibration curve created in an example of the present invention.

FIG. 2 is a graph showing α-galactosidase concentrations measured in (A) plasma and (B) serum in an example of the present invention.

FIG. 3 is a diagram showing the results obtained for significant differences by the Mann-Whitney test for (A) α-galactosidase concentrations in plasma for four groups: a group of male patients with classic Fabry disease, a group of male patients with atypical Fabry disease, a group of female Fabry heterozygous patients, and a group of healthy individuals; and (B) concentrations of α-galactosidase in serum for a group of male patients with classic Fabry disease and a group of healthy individuals.

### EMBODIMENTS FOR CARRYING OUT THE INVENTION

Unless otherwise noted in embodiments and examples, all procedures used are according to standard protocols such as M. R. Green & J. Sambrook (Ed.), Molecular cloning, a laboratory manual (4th edition), Cold Spring Harbor Press, Cold Spring Harbor, N.Y. (2012); and F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd., with or without modifications or changes. In addition, commercial reagent kits or measurement instruments are used as described in protocols attached thereto, unless otherwise noted.

The objects, features, advantages, and ideas of the present invention are apparent to those skilled in the art from the description of this specification. Those skilled in the art can easily reproduce the present invention from the description herein. The embodiments and specific examples described below represent preferable aspects of the present invention, which are given for the purpose of illustration or explanation. The present invention is not limited thereto. It is obvious to those skilled in the art that various changes and modifications may be made according to the description of the present specification within the spirit and scope of the present invention disclosed herein.

### ==Antibody in the MUSTag Form==

Antibody in the MUSTag form is described in, for example, the International patent publication No. WO 2010/001891 which is incorporated herein by reference.

Antibody in the MUSTag form is an antibody complex for detecting an antigen, which includes a nucleic acid chain used as a label, an anti-α-galactosidase antibody that specifically recognizes α-galactosidase to be detected, and an adaptor moiety that links the nucleic acid chain and the antibody, in which the adaptor moiety includes an immunoglobulin-binding domain of Protein G, Protein A, or Protein L.

The nucleic acid chain used as a label may be either a DNA or RNA, but DNA is preferable for easier detection. While the length of the nucleic acid chain is not specifically limited, shorter chains are preferable for enabling easy access of an enzyme to the chain for its cleavage or detection and nucleotides with a dozen to several tens of bases are also preferable for easier detection. Furthermore, the nucleic acid chain may be single-stranded or double-stranded, but it is preferable that the nucleic acid chain is double-stranded because of its stability. It is preferable that the nucleotide sequence of the nucleic acid chain is as specific as possible in order to detect it, for example, by PCR.

The nucleic acid chain and the antibody in the antibody complex are linked via the adaptor moiety. This ensures higher structural stability of the oligonucleotide-conjugated antibody and improves the yield of the complex obtained, leading to advantages such as higher detection sensitivity and better detection efficiency. The adaptor moiety is not specifically limited in its structure as long as it includes an immunoglobulin-binding domain of Protein G, Protein A, or Protein L. Therefore, the adaptor moiety may include either the protein itself of Protein G, Protein A, or Protein L or a fusion protein of their immunoglobulin-binding domain and another peptide. The Protein G, Protein A, or Protein L may be a wild-type protein or a mutant protein having an immunoglobulin binding ability.

For example, the immunoglobulin-binding domain of Protein G (GenBank accession number cDNA: X06173, protein: CAA29540) corresponds to the regions at the amino acid positions 303 to 357, 373 to 427 and 443 to 497. The immunoglobulin-binding domain of Protein A (GenBank accession number cDNA: M18264, protein: AAA26677) corresponds to the regions at the amino acid positions 39 to 88, 100 to 149, 158 to 207, 216 to 265, and 274 to 323. The immunoglobulin-binding domain of Protein L (GenBank accession number cDNA: M86697, protein: AAA25612) corresponds to the regions at the amino acid positions 115 to 173, 185 to 245, 257 to 317, 329 to 389, and 400 to 462.

The adaptor moiety may include a tag that is required when produced. The type of the tag is not specifically limited, and may be, for example, a GST-tag, an MBP-tag, a myc-tag or a flag-tag, but His-tag is preferable because it can bind to a small nickel molecule and therefore has no effect on chemical cross-linking.

The immunoglobulin-binding domain in the adaptor moiety is linked directly to the antibody, but may be either directly or indirectly linked to the nucleic acid chain. For indirect linking, for example, a biotin-binding domain of a biotin-binding protein and the immunoglobulin-binding domain of Protein G, Protein A, or Protein L are linked via a linker compound, or a fusion protein containing the two domains is formed. Other than these, various aspects can be contemplated, such as a case where the nucleic acid chain is conjugated with a biotin and the biotin then binds to a biotin-binding domain, or a case where each of the immunoglobulin-binding domain and the nucleic acid chain are linked to a biotin, and the biotins are linked to each other via a biotin-binding protein. For the linker compound, e.g., sulfosuccinimidyl 4-(N-maleimidomethyl)cyclohexane-1-carboxylate (Sulfo-SMCC) may be used.

The biotin-binding protein usually forms a homo-tetramer, and each subunit has one biotin-binding domain, i.e., a complete biotin-binding protein has four biotin-binding domains. The biotin-binding domain used in the MUSTag may have only one such subunit but may be tetrameric. It is preferable that one molecule of the nucleic acid chain binds to one molecule of Protein G to provide a structure enabling the nucleic acid chain used as a label to be exposed in solution. To make this structure, it is preferable to use a monomeric biotin-binding mutant protein with only one biotin-binding domain that does not form a tetramer. A monomeric biotin-binding mutant protein that maintains binding activity to biotin can be generated using, for example, a peptide that has the streptavidin amino acid sequence from positions 39 to 183.

It is noted that the biotin-binding protein includes, for example, avidin, streptavidin, and neutravidin. The biotin-binding domain corresponds to a region at amino acid positions 28 to 146 for avidin (RefSeq accession number cDNA: NM_{—}205320, protein: NP_{—}990651) and neutravidin which has the same sequence as avidin but is deglycosylated and a region at amino acid positions 39 to 156 for streptavidin (GenBank accession number cDNA: X03591, protein: CAA27265).

The adaptor moiety and the antibody may be chemically cross-linked, but the type of the cross-link is not specifically limited. Examples include cross-links between amino groups, those between carboxyl groups, and those between thiol groups. While the amino acid residue in the adaptor moiety which is to be cross-linked to the antibody is not specifically limited, it is preferable that the residue is the one within the immunoglobulin-binding domain directly bound to the antibody. Thus, in the antibody complex including a nucleic acid chain, an antibody and an adaptor moiety that links the nucleic acid chain and the antibody, the sensitivity for detecting an antigen is significantly enhanced by cross-linking the adaptor moiety and the antibody to form a cross-linked antibody complex.

The antibody may be a polyclonal antibody or a monoclonal antibody as long as it can specifically recognize α-galactosidase to be detected. The type of the antibody is not limited, and may be, for example, IgG or IgM. The antibody, however, needs to be able to bind to the immunoglobulin-binding domain included in the adaptor moiety.

The aforementioned antibody complex may include a cleavage site where the nucleic acid chain can be cleaved. The cleavage site may be provided in any one of the nucleic acid, the antibody and the adaptor moiety, but has different properties depending on the location where it is provided. For example, the cleavage site is cleaved by a restriction enzyme when provided in the nucleic acid, by a protease when provided in the antibody or a protein adaptor and by photo-irradiation or by active oxygen when a cross-linker (such as a divalent cross-linker) is provided as the adaptor. It is, however, preferable to provide a cleavage site cleaved by a restriction enzyme in the nucleic acid chain in terms of simplicity and specificity.

Furthermore, in order to make the nucleic acid chain to function as a label, a marker such as a radioactive isotope, a fluorescent dye, and an enzyme may be bound to the nucleic acid chain.

### ==Method of Producing an Anti-α-Galactosidase Antibody in the MUSTag Form==

A method of producing an antibody in the MUSTag form including a nucleic acid chain, an anti-α-galactosidase antibody, and an adaptor moiety that links the nucleic acid chain and the antibody is not specifically limited as long as the antibody in the MUSTag form having these components can be produced. For example, a method described in the International patent publication No. WO 2010/001891 can be used.

For example, the nucleic acid chain used as a label is linked to the adaptor moiety, the latter is fixed to the anti-α-galactosidase antibody to form an antibody complex, and then the adaptor moiety and the antibody are cross-linked using a chemical cross-linker.

When directly linked to the adaptor moiety, the nucleic acid chain may be linked to any position of the adaptor moiety. For example, it may be linked by modifying a terminal of the nucleic acid chain with an amino group or a thiol group and then chemically cross-linking the modified terminal to a functional group such as an amino group, a carboxyl group or a thiol group in the adaptor moiety using an appropriate cross-linker. When the nucleic acid chain is indirectly linked to the adaptor moiety, the link between the nucleic acid chain and the adaptor moiety may be achieved by linking the adaptor moiety to a biotin-binding domain of a biotin-binding protein, biotinylating the nucleic acid chain, and mixing them in a conventional method. Alternatively, the adaptor moiety and the nucleic acid chain may be biotinylated in advance and linked together via a biotin-binding protein by mixing them with the biotin-binding protein in a conventional method. Then, the adaptor moiety-nucleic acid chain complex may be mixed with the antibody in a conventional method to obtain the antibody complex.

Finally, by treating the antibody complex with a chemical cross-linker, the adaptor moiety and the antibody in the antibody complex can be cross-linked. Examples of the cross-linker include dimethyl pimelimidate, dimethyl suberimidate, and bis[sulfosuccinimidyl]suberate. It is preferable to use dimethyl pimelimidate to increase efficiency of cross-linking to thereby cross-link the antibody complex and the adaptor moiety with higher specificity.

### ==Method of Detecting α-Galactosidase==

Samples to be used for the detection of α-galactosidase as an antigen may be blood, serum, or plasma. For example, peripheral blood may be collected from a human to be diagnosed and processed using a known method to prepare a sample.

Alpha-galactosidase in the samples may or may not be immobilized on a support. For example, by immobilizing α-galactosidase on a support and allowing a cross-linked antibody complex to bind to the α-galactosidase, the cross-linked antibody complex can be linked to the support. The α-galactosidase may be immobilized on the support directly or indirectly, for example via an antibody. Subsequently, unreacted cross-linked antibody complex can be removed by washing the support with a buffer to obtain an ultra-pure antigen-antibody complex. The bottom surface of a plastic dish or beads may be used as the support, but beads are preferable because the background signal is lower. Commercially available beads for protein carriers, especially those for antibody carriers, such as magnetic beads and Sepharose beads, can be appropriately used.

To immobilize α-galactosidase on the support, the α-galactosidase may be linked to the support either directly or indirectly. For direct linking, a buffer containing α-galactosidase may be contacted with the support. For indirect linking, a substance such as an antibody, to which α-galactosidase can bind, may be bound to the support in advance, with which a sample containing α-galactosidase may be contacted. The latter indirect linking is preferable to increase specificity.

In order to detect the antibody in the MUSTag form bound to α-galactosidase, the nucleic acid in the antibody complex in the MUSTag form may be detected using PCR while keeping the nucleic acid in a container. For easier and more convenient detection, it is preferable to collect the nucleic acid.

The nucleic acid may be collected as the entire antibody complex. For example, the antigen and the antibody may be separated by a conventional method to collect the antibody complex. Alternatively, the nucleic acid alone may be collected. For example, an acid treatment, an alkaline treatment, a thermal treatment, or a protease treatment may be applied to denature or degrade the antibody complex in the MUSTag form.

Such an extreme treatment, however, does not make it possible to perform detection using an enzymatic reaction such as HRP, and thus the nucleic acid should be purified for PCR so that an enzyme such as Taq polymerase can work well. It is thus preferable that the cross-linked antibody complex has a cleavage site where the nucleic acid chain can be cleaved using a mild treatment such as a restriction enzyme treatment or a light treatment. In order to detect the nucleic acid chain, the nucleic acid chain is cleaved from the antigen-antibody complex at the cleavage site and collected. This collection step makes it possible to concentrate the nucleic acid chain before its detection, allowing detection of a smaller amount of the nucleic acid chain.

The nucleic acid chain thus collected is then detected. The detection method is not specifically limited. When a marker such as a radioisotope, a fluorescent dye or an enzyme is attached to the nucleic acid chain, this marker may be detected. It is, however, preferable to amplify and detect the nucleic acid chain because of the detection sensitivity. The amplification method used may be a conventional method such as PCR, LAMP and ICAN. The detection may be performed using a conventional method such as electrophoresis.

A standard curve is created in advance with an anti-α-galactosidase antibody in the MUSTag form using a method similar to the one described above with a standard product of α-galactosidase protein at known concentrations. While a method of using the anti-α-galactosidase antibody in the MUSTag form is not specifically limited, it is preferable that the collected nucleic acid chain is quantified by real-time PCR for accurate quantification.

### ==Method of Diagnosing Fabry Disease==

First, a sample to be examined is collected from a patient to be diagnosed. It is preferable to collect peripheral blood, as it is obtained relatively non-invasive methods. The collected blood may be processed to prepare serum or plasma. Alternatively, cells or a tissue from a patient can also be used. The type of cells or the tissue is not specifically limited, but leukocytes are preferred.

On the other hand, using the anti-α-galactosidase antibody in the MUSTag form adjusted as described above, a concentration of α-galactosidase in the sample of blood, serum, or plasma is measured.

For example, to diagnose a male patient with classic Fabry disease or a female homozygous patient with classic Fabry disease (both of whom are referred to as patients with classic Fabry disease), a threshold is set at 36 pg/mL or less, preferably 50 pg/mL or less, more preferably 78 pg/mL or less. A value smaller than the threshold can be considered to be indicative of a male patient with classic Fabry disease or a female homozygous patient with classic Fabry disease. In addition, to diagnose a male patient with atypical Fabry disease or a female homozygous patient with atypical Fabry disease, a threshold is set at 79 pg/mL or more, preferably 55 pg/mL or more as well as 520 pg/mL or less, preferably 1821 pg/mL or less. A value smaller than the threshold can be considered to be indicative of a male patient with atypical Fabry disease or a female homozygous patient with atypical Fabry disease. Furthermore, to diagnose a healthy individual without Fabry disease, a threshold is set at 3250 pg/mL or more, preferably 2950 pg/mL or more, more preferably 2000 pg/mL or more. A value smaller than this threshold can be considered to be indicative of a healthy individual without Fabry disease.

A patient with classic Fabry disease as used herein refers to a person having severe manifestations of the symptoms resulting from an almost complete absence of intracellular α-galactosidase activity due to deletion, insertion, nonsense mutation, or even some missense or splicing mutations of the α-galactosidase gene. A patient with atypical Fabry disease as used herein refers to a person who have residual α-galactosidase activity and mild manifestations of the symptoms due to other missense or splicing mutations. The patients with classic Fabry disease have disorders of various organs in the body, while those with atypical Fabry disease have disorders of a specific organ mainly such as the heart or kidney. Atypical Fabry disease is thus referred to as cardiac variant of Fabry disease or renal variant of Fabry disease according to the affected organ.

### ==Kit for Diagnosing Fabry Disease==

To make detection of α-galactosidase using the antibody in the MUSTag form easier, necessary reagents may be combined as a kit for diagnosing Fabry disease.

This kit includes an antibody complex including a nucleic acid chain used as a label, an antibody that specifically recognizes α-galactosidase, and an adaptor moiety that links the nucleic acid chain and the antibody, in which the adaptor moiety includes an immunoglobulin-binding domain of Protein G, Protein A, or Protein L, and the adaptor moiety and the antibody may be chemically cross-linked.

Other aspects of the antibody complex are as described above.

The kit may also comprise component(s) other than the cross-linked antibody complex and may include various buffers and reagents for detection such as primers and enzymes.

### EXAMPLES

The present invention is described more specifically in conjunction with Examples. The scope of the present invention is, however, not limited to the following Examples.

### [Method]

### (1) Production of an Anti-α-Galactosidase Antibody in the MUSTag Form

### (1-1) Production of Fusion Protein of Protein G/Streptavidin/His-Tag

First, a fusion protein of Protein G/streptavidin/His-tag (hereinafter, referred to as a fusion protein) was produced.

The following DNAs were chemically synthesized leaving the phosphate groups unprotected: DNA (SEQ ID No. 4, the nucleotide sequence at positions 1259 to 1381 in SEQ ID No. 3) encoding the amino acid sequence (SEQ ID No. 2) at positions 228 to 268 in the amino acid sequence set forth in SEQ ID No. 1 (full-length Protein G, GenBank accession number: M13825), which includes the region that binds to the Fc region of IgG antibody of Protein G; and DNA (SEQ ID No. 8, the nucleotide sequence at positions 164 to 598 in SEQ ID No. 7) encoding the amino acid sequence (SEQ ID No. 6) at positions 39 to 183 in the amino acid sequence set forth in SEQ ID No. 5 (full-length streptavidin, GenBank accession number: X03591), which includes the streptavidin region that binds to biotin. During this process, the double-stranded DNA fragments individually synthesized were ligated to prepare a full-length DNA. Subsequently, PCR was performed with the following primers using each of the synthesized DNAs as a template under the reaction conditions of 35 cycles of 95° C. for 30 sec., 55° C. for 30 sec., and 72° C. for 30 sec. to amplify the double-stranded DNAs. The primers were designed so that the DNA fragments obtained by PCR include recognition sites of restriction enzymes at their both ends (nucleotide sequences represented by small letters).

Protein G primer F:
(SEQ ID No. 9)
5′-CATATGCACTTACAAATTAATCCTTAA-3′
Protein G primer R:
(SEQ ID No. 10)
5′-GAATTCGGATCCTTCACCGTCAACACCGTTG-3′
streptavidin primer F:
(SEQ ID No. 11)
5′-GAATTCAAGCTTGCCGGCATCACCGGCACCTG-3′
streptavidin primer R:
(SEQ ID No. 12)
5′-CTGCAGCTGCTGAACGGCGTCGAGCG-3′

The DNA fragments thus obtained were digested with EcoRI, ligated together, and PCR was performed again using the Protein G primer F and the streptavidin primer R to amplify a fused DNA fragment.

Next, the amplified fused DNA fragment was digested with NdeI and inserted into an NdeI site of a bacterial expression vector pCR2.1 (Invitrogen) for synthesizing a fusion protein with His-tag. A recombinant vector including the nucleotide sequence (SEQ ID No. 14) encoding the fusion protein of Protein G/streptavidin/His-tag (SEQ ID No. 13) was thus constructed.

This recombinant vector was introduced into E. coli DH5α and the gene expression was induced by IPTG. The E. coli was then solubilized and the fusion protein of Protein G/streptavidin/His-tag was purified using Sepharose beads with immobilized nickel chelate (product name: Ni-NTA agarose, supplier company: QIAGEN, product No.: 30210).

### (1-2) Biotinylation of Oligonucleotide

An oligonucleotide chain #1 (SEQ ID No. 15) having a biotinylated 5′-end was synthesized by PCR under the reaction condition of 35 cycles of 95° C. for 60, 55° C. for 60 sec., and 72° C. for 30 sec. with the following primers (SEQ ID Nos. 16 and 17) including biotinylated primers (5-MUSTagBio) using, as a DNA template, pcDNA 3 (Invitrogen) into which DNA having the sequence (131 bp) of SEQ ID No. 15 had been inserted.

#1:
(SEQ ID No. 15)
5′-[Biotin]-CACTGCTTACTGGCTTATCGAAATGGAATTCTGCATGC
ATCTAGAGGGCCCTATTCTATAGCATAGTGTCACCTAAATGCTAGGCACC
TTCTAGTTGCCAGCCATCTGTTGCACACCAAACGTGGCTTGCC-3′

Likewise, an oligonucleotide chain #7 (SEQ ID No. 18) having a biotinylated 5′-end was synthesized with the same primers (SEQ ID Nos. 16 and 17) using, as a template, pcDNA 3 (Invitrogen) into which DNA having the sequence of SEQ ID No. 18 had been inserted.

#7:
(SEQ ID No. 18)
5′-[Biotin]-CACTGCTTACTGGCTTATCGAAATGGAATTCTGCATGC
ATCTAGAGGGCCCTATTCTATAGCATAGTGTCACCTAAATGCTAGGCAAC
CGACAATTGCATGAAGAACTCGCACATTGACGTCAATAATGACGTATGTT
CCCACCACCAAACGTGGCTTGCC-3′

### <Sequences of Primers for PCR>

5-MUSTag primer Bio-F:
(SEQ ID No. 16)
5′-[Biotin]-CACTGCTTACTGGCTTATCGAAA-3′
3-MUSTag primer R:
(SEQ ID No. 17)
5′-GGCAAGCCACGTTTGGTG-3′

### (1-3) Production of an Anti-α-Galactosidase Antibody in the MUSTag Form

243.4 μl of binding buffer (0.2 M Borate, pH 9.0, 0.5 M NaCl, 0.1 mM EDTA, 0.05% Monocaprate), 6.6 μl of fusion protein (100 pmol), 40 μl of biotinylated oligonucleotide (SEQ ID No. 15) (100 pmol) were added to a microcentrifuge tube, which was rotated at room temperature for 0.5 hours to bind the streptavidin region of the fusion protein and the biotinylated oligonucleotide. Subsequently, 60 μl of anti-α-galactosidase antibody (0.5 mg/ml) (200 pmol) was added and the tube was rotated at room temperature for 1 hour to bind the Protein G region of the fusion protein and the antibody.

DMP (Pierce, #21667, MW 259.177) adjusted to 6 mM with a coupling buffer just before use was added to an equal volume (about 350 μl) of the reaction solution and mixed together. The mixture was allowed to stand at room temperature for 1 hour. Then, 1 M Tris (pH 7.4) was added at a final concentration of 50 mM and the mixture was allowed to stand at room temperature for 15 minutes to stop the cross-linking reaction, after which the mixture was filtrated through a 0.45 μm PTFE filter (product name: Millex FH, supplier company: Millipore, product No.: SLFHR04NL). Finally, the reaction solution was fractioned by gel filtration chromatography under the conditions described below, and a peak fraction with the highest molecule weight was recovered as a MUSTag solution. The concentration of MUSTag in the solution was determined by comparing with the antibody used for the production of the MUSTag as a standard by ELISA.

### <Conditions for Gel Filtration Chromatography>

Equipment: product name: SMART system, supplier company: formerly Pharmacia (present GE Healthcare, a discontinued product)
Column: product name: Superdex 200 PC 3.2/30; supplier company: GE Healthcare; product No.: 17-1089-01

### Buffer: 10 mM Tris-HCl pH 7.4, 0.5 M NaCl, 0.1 mM EDTA, 0.05% Monocaprate

Flow rate: 100 μl/min.

### (2) Measurement of Concentrations of α-Galactosidase in Plasma Sample

(2-1) Detection of α-Galactosidase in Plasma and Serum Samples from Patients with Fabry Disease

Capture antibody (Anti-GLA mouse monoclonal antibody, clone No.: 4F2G4, IgG1) was attached to magnetic beads (Dynabeads M-280 Tosylactivated, Invitrogen) by coupling, to prepare 1% sensitized beads. The 1% sensitized beads were stored at 4° C. until measurement. The coupling procedure was performed according to manufacturer instructions of the protocol of Dynabeads M-280 Tosylactivated, Invitrogen.

Next, 25 μl of 1% sensitized beads and 40 μl (1 μg/mL) of the anti-α-galactosidase antibody in the MUSTag form were added to 4935 μl of MUSTag Beads Assay Buffer (0.05% Tween20, 0.45 M NaCl, 50 mM Phosphate Buffer (pH7.4), 10% Goat serum) to prepare a Beads/MUSTag mix solution.

Standards for calibrating the curve were obtained by diluting recombinant human α-galactosidase available from Genzyme (agalsidase beta, Fabrazyme (registered trademark)) used as a standard antigen with an antigen diluent (0.05% Tween20, 0.45 M NaCl, 50 mM Phosphate Buffer (pH7.4)) to final concentrations of 20,000; 4,000; 800; 160; 32; 6.4; and 1.28 pg/mL. The antigen diluent was used as a blank.

Next, 50 μL of the Beads/MUSTag mix solution was dispensed in each well of a 96-well U-bottom plate (cat No. 650001, Greiner), to which 50 μL of plasma or serum (diluted 1:10 using the antigen diluent) of patients with Fabry disease or standard was added. The reaction mixture was incubated while shaking at room temperature for 2 hours on a plate shaker. Each specimen was measured in triplicate.

After completion of the reaction, the plate was allowed to stand on a 96-well Magnetic-Ring Stand (Applied Biosystems) for 3 minutes and the supernatant was removed. After the removal of the supernatant, 200 μL of Wash Buffer (0.05% Tween20, 0.5 M NaCl, 20 mM Tris-HCl (pH7.4)) was added to each well and shaken on a plate shaker. The plate was allowed to stand on the 96-well Magnetic-Ring Stand for 3 minutes and the Wash Buffer was removed. This process was repeated four times to wash the magnetic beads.

After washing the plate, 30 μL of EcoRI restriction enzyme solution (EcoRI, NIPPON GENE) was added to the treated beads bound with the antigen-antibody in the MUSTag form, and the reaction mixture was incubated at room temperature for 15 minutes while shaking on a plate shaker. The plate was allowed to stand on the 96-well Magnetic-Ring Stand for 3 minutes and the supernatant was collected.

The collected EcoRI-reacted sample solution was analyzed by real-time PCR (real-time PCR reagent; FastStart Universal Probe Master (ROX), Roshe, real-time PCR system; Mx3005P, Stratagene) using a fluorescent probe [FAM; excitation wavelength of 492 nm/fluorescence wavelength of 516 nm]. The primers used were the oligonucleotides of SEQ ID Nos. 16 and 17 and the probe used was shown below:

(SEQ ID No. 19)
5′-[6-FAM]-CCTTCTAGTTGCCAGCCATCTGTT-[BHQ1]-3′.

### (2-2) Calculation of α-Galactosidase Concentration in Plasma and Serum

Nonlinear regression analysis was performed using GraphPad Prism version 5.02 (GraphPad Software Inc.) on Ct values determined by the real-time PCR to calculate concentrations of α-galactosidase in plasma and serum.

In the nonlinear regression analysis, the concentrations of the standard antigen used and the averages of Ct values obtained from the standard antigen were plotted on the horizontal (logarithmic) axis and the vertical (linear) axis, respectively, on a single logarithmic scale. Plots were fitted by regression using a four-parameter logistic model. The function of the curve was determined to take the following form.

Regression Equation of Calibration Curve (Four-Parameter Logistic Model)

$$y = {Ct}_{min} \frac{{Ct}_{max} - {Ct}_{min}}{1 + {10}^{( {LogEC}_{50} - LogX ) * Hill Slope}} ,$$

where
Ctₘₐₓ: theoretical maximum value of Ct (calculated by the least squares method)
Ctₘᵢₙ: theoretical minimum value of Ct (fixed to an average of the Ct values of blank)
EC₅₀: concentration at 50% response (calculated by the least squares method)
Hill Slope: steepness of the sigmoidal curve (calculated by the least squares method)

### [Results]

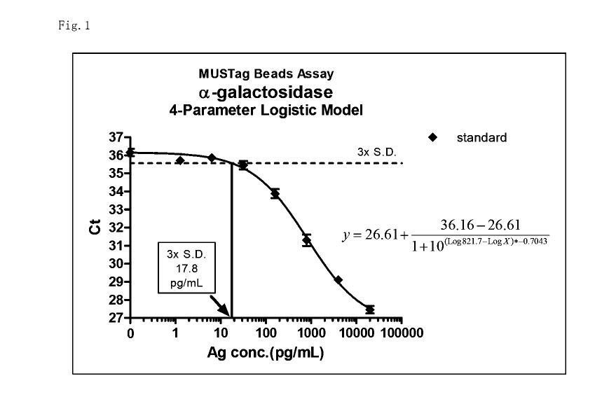

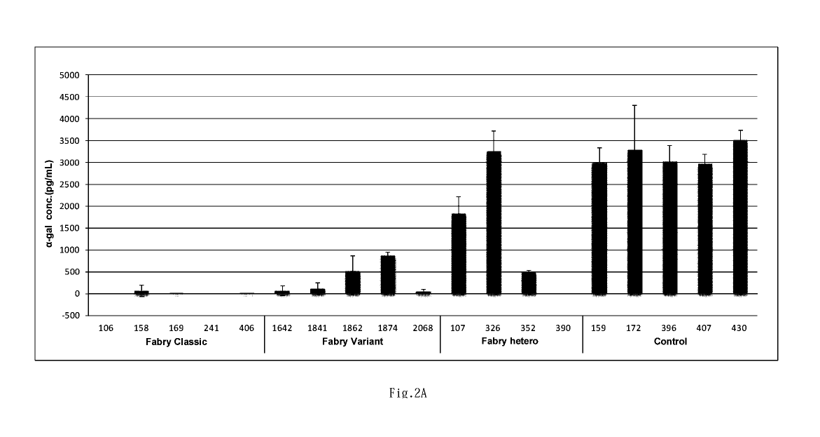

A calibration curve was produced using nonlinear regression analysis with Ct values of the standard human α-galactosidase samples determined by real-time PCR. The calibration curve is shown in FIG. 1. A detection limit of 3×SD was determined. The detection limit in this case was 17.8 pg/mL. The R-square of the calibration curve was 0.9946. Therefore, the values obtained by using this equation can be considered to be very close to real values.

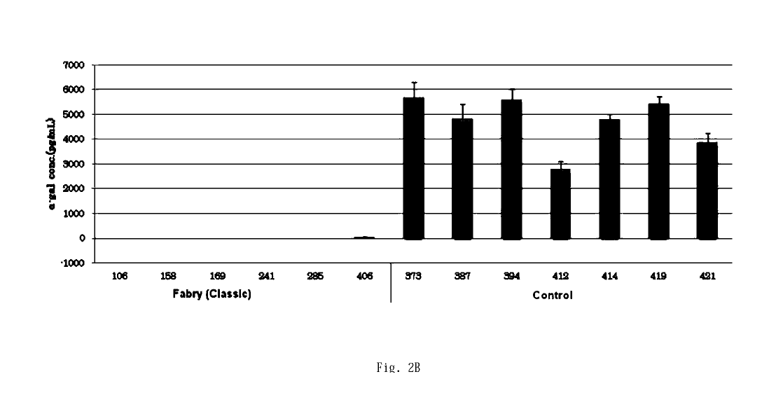

From the Ct values obtained by the real-time PCR, using the calibration curve equation in FIG. 1, concentrations of α-galactosidase in plasma or serum of patients with Fabry disease having various genotypes were calculated (Table 1 and FIG. 2).

Patients who clinically manifest symptoms of Fabry disease were diagnosed to have Fabry disease when a significant decrease was observed in measurements of α-galactosidase activity in their plasma. DNA was extracted from the blood of these patients, and a nucleotide sequence of the α-galactosidase gene was determined to genotype of each patient.

TABLE 1

| | | | GLA concentration | |
|---|---|---|---|---|
| | | | (pg/mL) | |
| Sample No. | | Genotype | Plasma | Serum |
| Classic | 106 | R227X | ND | ND |
| Fabry | 158 | W399X | 77 | ND |
| disease, | 169 | G195V | ND | ND |
| hemizygous | 241 | G43V | ND | ND |
| | 406 | Del2b | ND | 18.56 |
| | | (#718-719) | | |
| | 285 | ND | — | ND |
| Atypical | 1642 | Q279E | 79 | |
| Fabry | 1841 | IVS4 + 919G -> A | 127 | |
| disease, | 1862 | IVS4 + 919G -> A | 520 | |
| hemizygous | 1874 | IVS4 + 919G -> A | 880 | |
| | 2068 | A20P | 55 | |
| Fabry | 107 | R227X/WT | 1822 | |
| disease, | 326 | Del1b | 3240 | |
| heterozygous | | (#215)/WT | | |
| | 352 | R112C/WT | 493 | |
| | 390 | Y365/WT | ND | |
| Normal | 159 | WT/WT | 2984 | |
| | 172 | WT/WT | 3281 | |
| | 396 | WT/WT | 3007 | |
| | 407 | WT/WT | 2957 | |
| | 430 | WT/WT | 3499 | |
| | 373 | WT/WT | | 5654.8 |
| | 387 | WT/WT | | 4788.49 |
| | 394 | WT/WT | | 5559.86 |
| | 412 | WT/WT | | 2778.03 |
| | 414 | WT/WT | | 4769.88 |
| | 419 | WT/WT | | 5398.99 |
| | 421 | WT/WT | | 3855.24 |

ND: <17.8 pg/ml
blank cell: not measured

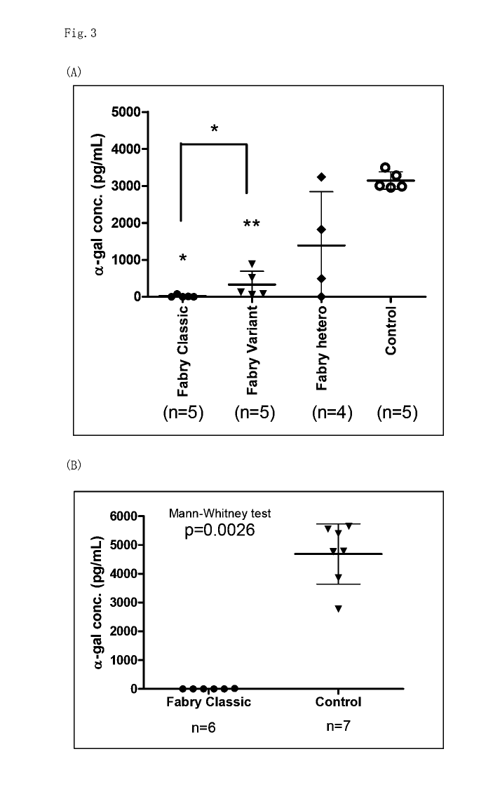

As shown in FIG. 3(A), the concentrations of α-galactosidase in plasma were investigated using the Mann-Whitney test. When the group of healthy individuals and the group of male patients with classic Fabry disease were compared, the p-value was 0.0119. When the group of healthy individuals and the group of male patients with atypical Fabry disease were compared, the p-value was 0.0079. When the group of healthy individuals and the group of female patients heterozygous for Fabry disease were compared, the p-value was 0.1111. When the group of male patients with classic Fabry disease and the group of male patients with atypical Fabry disease were compared, the p-value was 0.0212. And when the group of male patients with classic Fabry disease and the group of female patients heterozygous for Fabry disease were compared, the p-value was 0.1706. Accordingly, it was concluded that the concentrations of α-galactosidase in the plasma of the patients are different among the group of male patients with classic Fabry disease, the group of male patients with atypical Fabry disease, and the group of healthy individuals.

As shown in FIG. 3(B), the concentrations of α-galactosidase in serum were investigated using the Mann-Whitney test. When the group of healthy individuals and the group of male patients with classic Fabry disease were compared, the p-value was 0.0026. It was concluded that the concentrations of α-galactosidase in the serum of the patients are different between these two groups.

As described above, by measuring the concentration of α-galactosidase in blood, serum, or plasma using the MUSTag method, it is possible to distinguish between patients having classic Fabry disease (male hemizygous patients and female homozygous patients), patients having atypical Fabry disease (male hemizygous patients and female homozygous patients), and healthy individuals (wild-type).

1. A method of measuring a concentration of α-galactosidase in blood, serum, plasma, a cell, or a tissue, comprising the step of:
measuring the concentration of α-galactosidase using a MUSTag method; and
determining, using the measured concentration of α-galactosidase, whether the blood, serum, plasma, cell, or tissue is originated from a patient with classic Fabry disease, a patient with atypical Fabry disease or a healthy individual.

2. (canceled)

3. The method according to claim 1, wherein a concentration of saposin C is not measured in the blood, serum, plasma, cell, or tissue.

4. The method according to claim 1, wherein α-galactosidase activity is not measured in the blood, serum, plasma, cell, or tissue.
